(19) United States
(12) Patent Application Publication
(10) Pub. No.: US 2026/0146968 A1
(43) Pub. Date: May 28, 2026

(54) SYSTEMS AND METHODS FOR IDENTIFICATION OF IONS IN SOLUTION

(71) Applicant: TECHNION RESEARCH & DEVELOPMENT FOUNDATION LIMITED, Haifa (IL)

(72) Inventors: Ofer MANOR, Haifa (IL); Sudeepthi AREMANDA, Haifa (IL)

(21) Appl. No.: 19/448,431

(22) Filed: Jan. 14, 2026

Related U.S. Application Data
(63) Continuation of application No. PCT/IL2024/050756, filed on Jul. 31, 2024.

(30) Foreign Application Priority Data
Aug. 3, 2023 (IN) 202321052308

(51) Int. Cl. G01N 27/333; G01N 33/18

(52) U.S. Cl. CPC G01N 27/333; G01N 33/18

(57) ABSTRACT

The present invention relates to systems for identifying one or more ions in a liquid solution, including a solid surface configured to be in contact with the liquid solution; a mechanical wave generator configured to be in contact with the solid surface and/or with the liquid solution; and a working microelectrode configured to be positioned in the liquid solution in close proximity to the solid surface without contacting it. Methods of the invention include generating a mechanical wave which propagates and excites ions of an electrical double layer (EDL) formed at an interface between the solid surface and the liquid solution, thereby causing the EDL to vibrate and to leak an electrical field. The electrode measures electrical potential of the electrical field over time, which yields a voltage spectrum comprising amplitude and phase which are indicative of an identity and a concentration of the one or more ions in the liquid solution.

### CROSS REFERENCE TO RELATED APPLICATIONS

This application is a Bypass Continuation of PCT Patent Application No. PCT/IL2024/050756 having International filing date of Jul. 31, 2024, which claims the benefit of priority of Indian Patent Application No. 202321052308, filed Aug. 3, 2023, the contents of which are all incorporated herein by reference in their entirety.

### TECHNICAL FIELD

The present disclosure generally relates to identifying ions in liquid solutions, and more particularly, to systems and methods for ultrasonic analysis of the presence of ions and their concentration in solutions.

### BACKGROUND

Most current methods for identifying the presence of ions in water, e.g., ion chromatography, titration, and FTIR, are defined for lab use, require benchtop equipment, generally priced in a range of several thousands to hundreds of thousands of dollars, and trained personnel. Ion-selective electrodes are another common method for identifying ions in solutions. It is relatively flexible in use and in size. However, the use of ion-selective electrodes also has the downsides of requiring trained personnel and can identify only a single ion species per a specific electrode surface.

Ions in electrolyte solutions accumulate near charged surfaces in a form known as the electrical double layer (EDL). This is a surface phenomenon and is comprised from two charged layers. The charged surface layer may be comprised from a static layer of ions chemically or physically attached to the surface. The surface may be charge-inert otherwise or may be connected to an external power source, e.g., a battery, that contributes to the electrical potential on the solid surface. A second charge layer is usually of a thickness of half a nanometer to hundreds of nanometers in the electrolyte solution away from the surface. It is comprised from a dense atmosphere of ions, in particular counter charged ions (relatively to the concentration of ions in the electrolyte solution) and is known as the diffusive ion layer. At equilibrium, this second layer screens the electrical field emanating from the charged surface layer into the electrolyte solution. These ionic structures are fundamental across countless natural and artificial systems and support many applications. Examples for electrokinetic applications include electrophoretic particle motion, electroosmotic actuation of flow, and electrowetting contributions to liquid film and drop dynamics. Examples for equilibrium applications include energy barriers to particulate coagulation and adsorption, guiding folding structures of proteins, and surface electrical properties of biological and artificial membranes.

Accordingly, measuring features of EDLs may be applied in order to identify ions in solutions and measure their concentrations.

Many experimental techniques have been employed to examine the structure and properties of EDLs in their equilibrium or electrokinetic steady state. These include impedance spectroscopy, nuclear magnetic resonance spectroscopy, ambient pressure X-ray photoelectron spectroscopy, spectroscopy, ambient pressure X-ray photoelectron spectroscopy, electrochemical quartz crystal microbalance, electrical potential microscopy, infrared vibrational spectroscopy (e.g., Fourier-transform infrared spectroscopy (FTIR) and atomic force microscopy. Moreover, zeta potential analyzers employ electrophoresis and electroacoustics in dilute and dense particle suspensions, respectively, or streaming potential/current near flat substrates to measure steady or quasi-steady EDL properties.

Therefore, while EDLs have a potential for applications such as measuring ion concentrations, there is a need for a simple-to-use method which does not require expertise and expensive laboratory equipment for identifying ions in solutions and measuring their concentrations.

### SUMMARY

Aspects of the disclosure, according to some embodiments thereof, relate to identifying ions in liquid solution, and more particularly, to a system and a method for identifying ions and mixtures thereof based on analyzing the electrical properties thereof.

According to some embodiments, there is provided herein a system for identifying one or more ions in a liquid solution, the system including:
a solid surface configured to be in contact with the liquid solution;
a mechanical wave generator configured to be in contact with the solid surface and/or with the liquid solution and to generate a mechanical wave, the mechanical wave being configured to propagate and excite ions in an electrical double layer (EDL) formed at an interface between the solid surface and the liquid solution, thereby causing the ions of the EDL to vibrate and to emanate an electrical field away therefrom; and
a working microelectrode positioned configured to be in the liquid solution in close proximity to the solid surface without contacting it, and to measure electrical potential resulting from the electrical field emanating from the EDL over time.

In some embodiments, the electrical potential, when measured over time, yields a voltage spectrum, including amplitude and phase which are indicative of an identity and a concentration of the one or more ions in the liquid solution.

In some embodiments, the solid surface forms part of the mechanical wave generator. In some embodiments, the solid surface is made of or includes one or more metals, one or more ceramic materials, one or more polymers, glass, or any combination thereof. In some embodiments, the solid surface is a piezoelectric solid surface. In some embodiments, the solid surface is made of or includes at least one of lithium niobate, lead zirconate titanate, amorphous or crystalline silicon oxide, glass, plastic, and combinations thereof.

In some embodiments, the mechanical wave generator is directly or indirectly attached to the solid surface. In some embodiments, the mechanical wave generator is selected from an acoustic actuator, a mechanical actuator, an ultrasonic actuator, a piezoelectric actuator, a surface acoustic wave actuator, and a surface acoustic wave device.

In some embodiments, the mechanical wave is selected from a surface or surface acoustic wave (SAW), a lamb wave, a shear wave, a flexural wave, and a Love wave, and combinations thereof. In some embodiments, the surface wave or the SAW is selected from a Rayleigh wave, a leaky Rayleigh wave, a Scholte wave, a Sezawa wave, and combinations thereof. In some embodiments, the mechanical wave is a standing wave. In some embodiments, the mechanical wave has a frequency of about 1 KHz to about 1 GHz. In some embodiments, the mechanical wave includes at least one wave having a frequency which is close to a relaxation frequency or to half the relaxation frequency, of the one or more ions, or close to the relaxation frequency multiplied by an integer. In some embodiments, the mechanical wave includes a single wave. In some embodiments, the mechanical wave includes a plurality of waves having different frequencies and/or amplitudes.

In some embodiments, the mechanical wave generator includes a frequency modulator for temporally modulating the frequency of the mechanical wave throughout the measuring of the electrical potential over time. In some embodiments, the mechanical wave generator includes an amplitude modulator for temporally modulating the amplitude of the mechanical wave throughout the measuring of the electrical potential over time.

In some embodiments, the working electrode is positioned in the liquid solution at a distance of about 1-200 micron from the solid surface. In some embodiments, the system further includes a reference electrode. In some embodiments, the reference electrode is positioned in the liquid solution further away from the solid surface than the working electrode, and at least about 200 micron, 500 micron, or 1 millimeter away from the solid surface.

In some embodiments, the system further includes a processor including a computation module configured to analyze a spectrum of the electrical potential measured over time and to provide an identify and/or a concentration of the one or more ions.

In some embodiments, there is provided herein a method for identifying one or more ions in a liquid solution, the method including:
a) contacting the solution with a solid surface;
b) generating, by a mechanical wave generator configured to be in contact with the solid surface and/or with the liquid solution, a mechanical wave including at least one frequency and at least one amplitude, which propagates and excites ions in an electrical double layer (EDL) formed at an interface between the surface and the solution, thereby resulting in the EDL emanating an electrical field away therefrom;
c) measuring, by a working microelectrode configured to be positioned in the liquid solution in close proximity to the solid surface without contacting it, for each frequency and/or amplitude in the mechanical wave, a voltage of the electrical field emanated from the EDL over time; and
d) analyzing, by a computation module in a processor, a spectrum of the voltage measured for frequencies and/or amplitudes included in the mechanical wave to identify the one or more ions and concentrations thereof in the solution.

In some embodiments, the voltage spectrum includes amplitude and phase indicative of the identity and the concentration of the one or more ions in the liquid solution.

In some embodiments, contacting the liquid solution with the solid surface in step (a) includes placing a drop of the liquid solution on the solid surface. In some embodiments, contacting the liquid solution with the solid surface in step (a) includes at least partially immersing the solid surface in the liquid solution.

In some embodiments, generating a mechanical wave in step (b) includes generating a plurality of waves including a plurality of different frequencies and/or a plurality of different amplitudes. In some embodiments, generating a mechanical wave in step (b) includes temporally sweeping through a range or a plurality of frequencies, and for each frequency measuring in step (c) the voltage leaked from the EDL. In some embodiments, generating a mechanical wave in step (b) includes temporally modulating the mechanical wave amplitude, and for each amplitude measuring in step (c) the voltage leaked from the EDL. In some embodiments, generating a mechanical wave in step (b) includes temporally modulating at least one concentration of the one or more ions.

In some embodiments, analyzing the voltage spectrum in step (d) includes identifying at least one peak intensity of the voltage spectrum that is induced by a certain wave frequency, and identifying the one or more ions based on the ion having a relaxation frequency, half a relaxation frequency, or a relaxation frequency multiplied by an integer, that is close to the certain frequency. In some embodiments, analyzing the voltage spectrum in step (d) further includes identifying a series of peak amplitudes or intensities of the voltage spectrum, including a main peak at a wave frequency corresponding to a leading ion resonance harmonic and at least one smaller peak corresponding to secondary harmonics.

In some embodiments, the method further includes adding at least one known ion at a known concentration to the liquid solution prior to generating the mechanical wave in step (b).

In some embodiments, the method further includes performing steps (a)-(c) on at least one control solution including at least one known ion at a known concentration, and wherein the analyzing in step (d) includes adjusting the voltage spectrum obtained for the liquid solution based on the voltage spectrum obtained for the control solution. In some embodiments, the at least one known ion includes sodium and/or chlorine ions. In some embodiments, the at least one known ion includes a known salt.

In some embodiments, the one or more ions are two or more ions. In some embodiments, the one or more ions include biologically harmful/heavy metal ions; and/or common water ions. In some embodiments, the biologically harmful/heavy metal ions are selected from nitrates, lead, arsenic, selenium, cadmium, and any mixtures thereof, and/or the common water ions are selected from sodium, potassium, chlorine, calcium, magnesium, and any mixtures thereof. In some embodiments, the one or more ions include at least one ion present at a concentration of at least about 1 ppm.

In some embodiments, the liquid solution is selected from fresh water, saline water, tap water, pool water, water in pipes, sea water, ocean water, river water, reservoir water, waste water, sewage water, biological fluids, bodily fluids, water used for industrial processes/facilities, or any combination thereof.

In some embodiments, zeta potential is further computed based on the measured voltage.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not drawn to scale. Moreover, two different objects in the same figure may be drawn to different scales. In particular, the scale of some objects may be greatly exaggerated as compared to other objects in the same figure.

In block diagrams and flowcharts, optional elements/components and optional stages may be included within dashed boxes.

In the figures:

FIGS. 1A-1D provides a description of the theory. FIG. 1A. An illustration of boundary layer flow streamlines, φ, in an electrolyte solution of positive and negative ions (+, −) next to a charge solid, releasing an electrical field, ψ, to the solution. FIG. 1B. A theory describing temporal (t) variations of the measured voltage in the presence (ψ1) and absence (ψair) of an electrical double layer (EDL), following the application of a voltage signal, ψi, to the piezoelectric actuator. The numbers in parentheses (0.1, 1, 10) indicate he ratios between the diffusion coefficients of two different ions in the solution.

FIGS. 1C-1D. tr(m+) variations of the measured voltage amplitude, |ψ1| (FIG. 1C), and phase, ΔΘ (FIG. 1D), for different tr(m+)/tr(m−) ratios (0.1—dashed red, 0.5—dashed blue, 1—black, 5—blue, and 10—red) and for β=ψs=ψi=1, where tr(m+) and tr(m−) are the characteristic acoustic to EDL time ratios for positive and negative ion species (m+, m−), respectively, and where tr≡tr(m+)=tr(m−) for same ion diffusion coefficients. In the inset we assume β=ψs=tr(m+)=tr(m−)=1 to show that the |ψ1| maxima, marked with circles, migrate to greater tr(m+) values when increasing |ψi|.

FIG. 2 shows a schematic illustration of an exemplary system according to some embodiments of the invention. Dashed lines represent optional embodiments.

FIGS. 3A-3B show: FIG. 3A. Fourier transform (FFT) of the electrical potential leakage in NaNO₃ solution with (black line) and without (red line) SAW-excitation; the inset shows the voltage signal applied to the SAW device (by amplitude modulation, blue line) and the response of the EDL (black line). FIG. 3B. Variation in the magnitude of electrical potential leakage (ψ₁) with Ionic strength (I) and acoustic to EDL time ratio and tᵣ for NaNO₃ solution tᵣ(Na⁺) at various power levels (by amplitude modulation) of SAW-excitation (at the voltages of 100-1000 mV, as indicated in the legend).

FIGS. 4A-4B show plots of scaled electrical potential leakage (ψ₁, upper panel) and phase difference (lower panel) between the excitation SAW signal and ψ₁ plotted against ionic strength (I) and tᵣ for NaNO₃ (FIG. 4A) and KCl (FIG. 4B) solutions at various power levels of SAW-excitation (by amplitude modulation, at voltages of 20-100 mV, as indicated in the legend). (note: the dotted circles and their respective inset figures indicate the shift of maxima of |ψ₁|/|ψᵢ| to increasingly larger values of tᵣ(m+) as the applied voltage level |ψᵢ| is increased in line with theoretical predictions shown in FIG. 1C).

FIGS. 5A-5B show plots of scaled electrical potential leakage (ψ₁, upper panel) and phase difference (lower panel) between the excitation SAW signal and ψ₁ plotted against ionic strength (I) and tᵣ for NaCl (FIG. 5A) and MgSO₄ (FIG. 5B) solutions at various power levels (by amplitude modulation, at voltages of 20-100 mV, as indicated in the legend) of SAW-excitation. (note: the dotted circles and their respective inset figures indicate the shift of maxima of |ψ₁|/|ψᵢ| to increasingly larger values of tᵣ(m⁺) as the applied voltage level |ψᵢ| is increased in line with theoretical predictions shown in FIG. 1C).

FIGS. 6A-6C show: FIG. 6A. Comparison of the experimental path-length for ion motion with the theoretical at varying ionic strength values (I). FIG. 6B. Scaled electrical zeta potential leakage (ψ₁) plotted against tᵣ for NaNO₃ solutions at pH: 4.1, 6, and 9.6. FIG. 6C. Variation in the mean of the scaled electrical potential leakage (|ψ₁ₘₑₐₙ|) with zeta potential at various power levels (by amplitude modulation, at voltages of 20-100 mV, as indicated in the legend) of SAW-excitation.

### DETAILED DESCRIPTION

The principles, uses and implementations of the teachings herein may be better understood with reference to the accompanying description and figures. Upon perusal of the description and figures present herein, one skilled in the art will be able to implement the teachings herein without undue effort or experimentation.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

In the following description, various aspects of the invention will be described. For the purpose of explanation, specific details are set forth in order to provide a thorough understanding of the invention. However, it will also be apparent to one skilled in the art that the invention may be practiced without specific details being presented herein. Furthermore, well-known features may be omitted or simplified in order not to obscure the invention.

The present invention relates to systems and methods for ultrasonic analysis of the presence of ions and their concentration and for the analysis of static and dynamic electrical properties of charged ions surfaces, in solutions.

The present invention is based on the finding that an electrical field leaked, or emanated, from a surface acoustic wave (SAW)-excited electrical double layer (EDL), discloses intimate dynamic and static properties of the EDL, and particularly the presence of electromechanical ion resonance under periodic excitation. Ion-specific electrical leakage magnitude and phase spectra are distinctive of the ion species and concentrations and correlate with the zeta potential of the charged surface. These findings are the basis of the novel approach presented hereinbelow, which facilitates acquiring dynamic electrical properties of surfaces and characterize electrolyte solutions using the systems and methods described herein.

The inventors measured ion dynamics in EDLs by applying a SAW in a solid substrate in contact with a drop of an electrolyte solution. Recent evidence suggest that SAWs interfere with EDLs, thus generating electrokinetic effects in applications of catalysis and liquid film dynamics (Ahmed et al., Nature Commun. 10, 2282 (2019); Rezk et al., Phys. Chem. Lett. 11(12), 4655-4661 (2020); Dietzel et al., Phys. Rev. Fluids 5, 123702 (2020); Horesh et al., Phys. Rev. Fluids 5, 114002 (2020); Morozov et al., Curr. Opin. Colloid Interface Sci. 36, 37-45 (2018)). A traveling SAW, a nanoscale reminiscent of the Rayleigh component in seismic waves, generated a same frequency spatiotemporal periodic boundary layer flow near the solid surface within a viscous penetration length of δ=\sqrt{p2u/ρω}≈10⁻⁷ m, where μ, ρ and ω are the shear viscosity, the density of ambient water, and the angular frequency of a 20 MHz-frequency SAW, respectively.

The length and time scale of the SAW induced boundary layer flow that may be comparable to the intrinsic ones of the EDL for a corresponding parameter group, including the diffusion coefficients, D(mᵢ), of ions, mᵢ, and the ionic strength,

$$I = {\sum }_{i = 1} {c}_{i} {z}_{i}^{2} / 2 ,$$

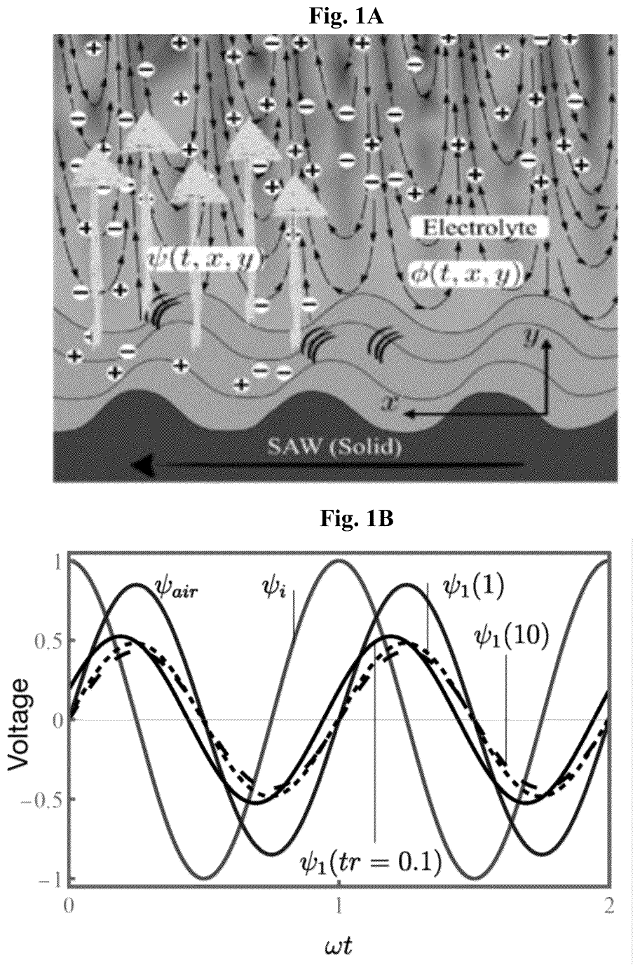

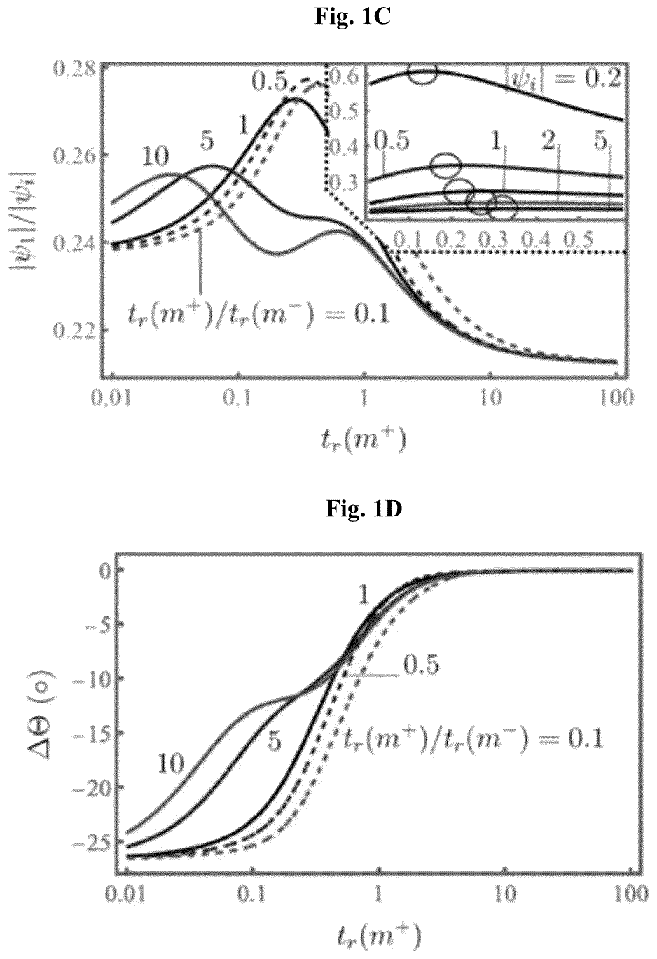

of the electrolyte solution; the ionic strength sums over the contributions of ion concentration (cᵢ) and valence (zᵢ) to electrical field screening by the EDL. The EDL Debye length, σ≡\sqrt{ϵkBT/2e²I}, was in the range of 1-670 nanometer in ambient conditions for I=10⁻¹-10⁻⁷ Molar (gram-mole/liter), corresponding to physiological saline water and deionized water at pH 7, respectively, where ϵ, k_{B} and T are the permittivity of the electrolyte solution, Boltzmann constant, and temperature. The respective natural relaxation time of a small ion species mᵢ in an EDL is in the range of σ²/D(mᵢ)=10⁻⁶-10⁻⁹ s, assuming that D(mᵢ)=10⁻⁹ m²/s. Should the acoustic length (δ) and time (ω⁻¹) scales overlap with the EDL Debye length (σ) and ion relaxation time, respectively, it would be expected that the EDL and the SAW would become dynamically entangled. A recent theory (Dubrovski and Manor, Langmuir 37(50), 14679-14687 (2021) suggest that such an electrokinetic entanglement would leak an electrical field off the EDL, including information about static and dynamic EDS properties. The theoretical basis for the invention is explained in more detail below, with reference to FIG. 1.

In the present work, the inventors show that measuring the electric field leaked off of SAW-excited EDL can further be used for identifying ion species in solution, and measuring their concentration.

The methods and systems of the invention facilitate simultaneous detection of one or more ion species or mixtures thereof in electrolyte solutions for domestic residentials and public and industrial facilities for water quality assessment. The use of the system/method may be in the form of a continuous method to simultaneously identify the presence of multiple ion species in water with the aim of identifying polluting and biologically harmful ions, such as nitrates and lead, alongside common ions in water, such as sodium, potassium, and chlorine, down to 1 ppm (particle per million) resolution on one detection-surface and advantageously without using ion sensitive barriers (as in prior art ion-selective electrodes) for integration in the water systems of domestic residentials, public and industrial facilities, and laboratories, for water quality assessment, without the requirement for trained personnel.

The systems and methods disclosed herein further provide the ability to measure zeta potential of flat surfaces and rate of charge and discharge of the electrical double layer on a flat surface. This provides a method that is simpler and cheaper than existing methods for analyzing these properties, such as stream current and stream potential measurements.

Advantageously, the disclosed herein systems and methods are technically simple to operate in a continuous manner.

Advantageously, the disclosed herein systems and methods may be integrated in water systems (water meters, taps) of domestic residentials and public and industrial facilities for water quality assessment, i.e., to identify that necessary ions at corresponding concentrations exist in water and to identify the presence of unwanted ions, harmful to humans or vegetation.

Advantageously, the disclosed herein systems and methods allow for further measuring static and dynamic electrical properties of surfaces, i.e., zeta potential of the surface and ion relaxation time in the electrical double layer using a procedure much simpler and much cheaper than the currently existing procedures using streaming current and potential.

### Systems for Identifying Ions in Solution

According to some embodiments, there is provided herein a system for identifying one or more ions in a liquid solution, the system including:
a solid surface configured to be in contact with the liquid solution;
a mechanical wave generator configured to be in contact with the solid surface and/or with the liquid solution and to generate a mechanical wave, the mechanical wave being configured to propagate and excite ions in an EDL formed at an interface between the solid surface and the liquid solution, thereby causing the ions in the EDL to vibrate and to emanate an electrical field away therefrom; and
a working microelectrode configured to be positioned in the liquid solution in close proximity to the solid surface without contacting it, and to measure electrical potential resulting from the electrical field emanating from the EDL over time.

In some embodiments, the electrical potential measured over time yields a voltage spectrum including amplitude and phase which are indicative of an identity and a concentration of the one or more ions in the liquid solution.

The term “vibrate”, as used herein, relates to mechanical vibrations of ions in the EDL, i.e., to that ions mechanically displace in a periodic manner.

The terms “voltage” and “electrical potential” are used herein interchangeably.

The phrase “over time” is not meant to include any specific amount of time, but rather to indicate that the measurement is performed over time, so as to allow a voltage spectrum to be generated including a variety of voltages. The voltage spectrum is analyzed according to the invention, as detailed below. The time may encompass several seconds, several minutes, or several hours, etc.

The working electrodes is “configured to be positioned in the liquid solution” since it is actually positioned in the liquid solution only when the solution is added to the solid surface.

The term “in contact” as used herein with reference to the mechanical wave generator, means that the mechanical wave generator may directly touch the solid surface and/or the liquid solution, or the mechanical wave generator may be in contact with the solid surface and/or the liquid solution through additional solid and/or liquid and/or fluid phases that assist in transmitting the mechanical wave from the generator to its destination in the solid or in the liquid solution. In other words, the contact between the wave generator and the solid surface and/or liquid solution may be direct or indirect contact.

It is noted that the term “ion”, as used herein, relates to ion species and not to single ion entities. Accordingly, different ions relate to different ion species, such as Na⁺, K⁺, Cl⁻, etc. Sometimes the term “ion species” is explicitly used.

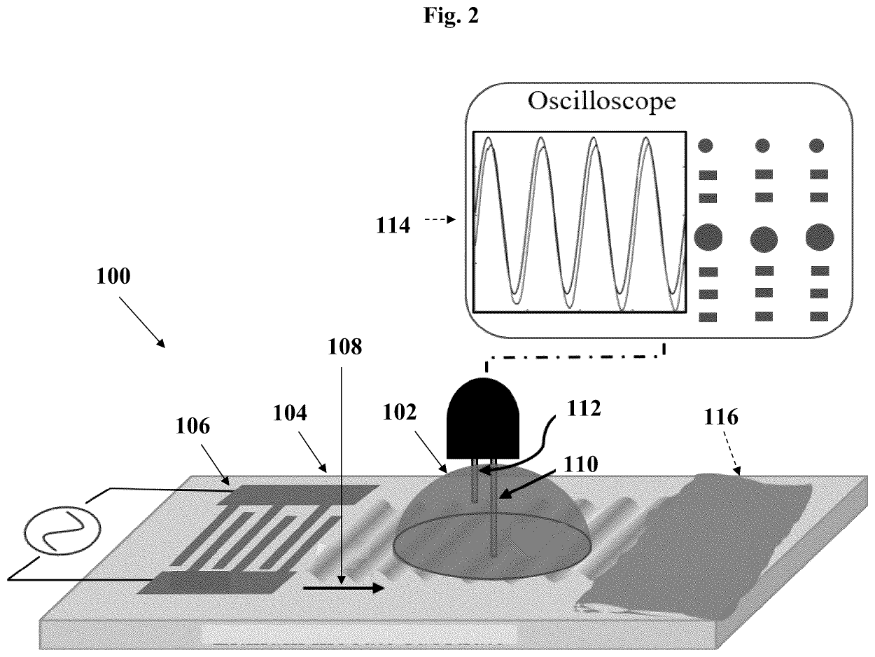

Reference is made to FIG. 2, which shows a schematic illustration of exemplary system 100, according to some embodiments. In this case, drop 102 of an electrolyte solution is placed atop surface 104, which may be a surface of a substrate, e.g., a Lithium niobate substrate. It is noted that drop 102 may also be a part of a liquid solution in which system 100 is immersed. The surface is associated with wave generator 106, such as a piezoelectric actuator optionally including aluminum interdigital transducer (IDT). Wave generator 106 generates wave 108, which may be a traveling SAW, to measure the electrical potential (or voltage spectrum) emanating from the solid/solution interface by placing working electrode 110 in the solution, at close proximity to the solid surface, such as about 40 μm away therefrom, and not touching the surface. The measurement may optionally be conducted against reference electrode 112, which is positioned in the solution, further away from the solid surface, such as at the bulk of drop. The electrodes are optionally connected to oscilloscope 114 for obtaining frequency variation. Optionally, in case a drop is used (rather than immersing the system in the liquid solution), water-based acoustic-wave absorber 116 may be placed next to the drop.

According to some embodiments, the one or more ions (ion species) are more than one ion species, such as at least 2, 3, 4, or 5 ion species. According to some embodiments, the one or more ions are two or more ions (i.e., different ion species).

According to some embodiments, the one or more ions do not include all of the ions present in the liquid solution. According to some embodiments, the one or more ions include all ions present in the liquid solution.

According to some embodiments, the one or more ions include one or more of: biologically harmful ions, heavy metal ions, and common water ions.

According to some embodiments, the biologically harmful ions and/or the heavy metal ions include one or more of: nitrates, lead, arsenic, selenium, cadmium, and any mixtures thereof.

According to some embodiments, the common water ions include one or more of: sodium, potassium, chlorine, calcium, magnesium, and any mixtures thereof.

According to some embodiments, the system is configured to identify the one or more ions when present in the liquid solution at a concentration of at least about 1 ppm (e.g., 1 milligram per liter). According to some embodiments, the system is configured to identify the one or more ions when present in the liquid solution at a concentration of at least about 10⁻⁵ M.

According to some embodiments, the system is configured to identify the one or more ions when present in the liquid solution at a concentration of at least about 1 ppm, or at least about 10⁻⁶M, or about 10⁻⁶M-10⁻⁵M).

The liquid solution may be any electrolyte solution for which identifying ions or measuring their concentration is desired. According to some embodiments, the liquid solution includes water. According to some embodiments, the liquid solution includes any of fresh water, saline water, tap water, pool water, water in pipes, sea water, ocean water, river water, reservoir water, waste water, sewage water, biological fluids, bodily fluids, water used for industrial processes/facilities, or any combination thereof.

According to some embodiments, the liquid solution is in the form of a drop (or drops), such as drop 102. In some embodiments, the drop is positioned on the solid surface.

The term “drop”, as used herein, generally relates to a small amount of the liquid solution which is not sufficient to cover the solid surface. Typically, this term relates to a small amount of liquid, such as a droplet. In some embodiments, the size of a drop is less than about 1 ml. In some embodiments, the size of a drop is less than about 900, 800, 700, 600, or 500 microliter.

According to some embodiments, the solid surface is part of the mechanical wave generator.

According to some embodiments, the solid surface is a coating of the mechanical wave generator.

According to some embodiments, the solid surface is a layer of adsorbed molecules or molecular ligands on the mechanical wave generator.

According to some embodiments, the solid surface is part of a solid substrate, such as shown for solid surface 104 in FIG. 2.

According to some embodiments, the solid substrate is capable of enabling propagation of the mechanical wave therethrough.

According to some embodiments, the solid substrate or the solid surface is made of or includes one or more metals, one or more ceramic materials, one or more polymers, glass, or any combination thereof.

According to some embodiments, the glass is made of or include one or more of: soda-lime, borosilicate, aluminosilicate, and lead silicate.

According to some embodiments, the solid surface or the solid substrate is made of or includes polymethyl methacrylate and/or polyethylene.

According to some embodiments, the solid surface is non-piezoelectric.

According to some embodiments, the solid surface is piezoelectric.

According to some embodiments, the piezoelectric solid used to generate the mechanical wave is made of or includes one or more of: piezoelectric lithium niobate, piezoelectric lead zirconate titanate (PZT), piezoelectric silicon oxide (quartz), piezoelectric aluminum nitride, piezoelectric lithium tantalate, piezoelectric polyvinylidene fluoride, piezoelectric barium titanate, and piezoelectric zinc oxide.

According to some embodiments, the solid surface is at least partially immersed in the liquid solution.

According to some embodiments, the solid substrate has mechanical properties enabling it to support the mechanical wave at the frequency range thereof.

As a non-limiting example, the solid substrate may be made of or include lithium niobate, lead zirconate titanate (PZT), amorphous or crystalline silicon oxide, glass (e.g., soda-lime, borosilicate, aluminosilicate, lead silicate), plastic (e.g., polymethyl methacrylate, polyethylene, etc.).

In some embodiment, the solid substrate is made of a negatively charged material. In some embodiment, the solid substrate is made of a positively charged material.

According to some embodiments, the solid surface is substantially flat near the working electrode.

According to some embodiments, the substantially flat solid surface is devoid of an average curvature of the solid surface near the working electrode.

According to some embodiments, the solid surface has an area of at least about 10000 squared micrometers.

According to some embodiments, the solid surface is macro-scale (e.g., larger than about 10,000 squared micrometers) and substantially flat (i.e., substantially devoid of an average curvature).

The mechanical wave generator, such as wave generator 106, generates a mechanical wave which propagates and mechanically excites (vibrates) ions of an EDL formed at an interface between the solid surface and the liquid solution. Accordingly, the wave generator may be attached to the solid surface and not necessarily directly contact the liquid solution. This is, since the wave propagates through the solid surface and reaches the EDL. Alternatively, the wave generator may contact the liquid solution without directly contacting the solid surface, and the wave propagates through the liquid solution and reaches the EDL. It is also possible that the wave generator contacts both the solid surface and the liquid solution. As noted above, another option is that the wave generator indirectly contacts the solid phase and/or the liquid solution via mediating layers or phases.

According to some embodiments, the mechanical wave generator is attached to the solid surface. According to some embodiments, the mechanical wave generator is directly attached to the solid surface. According to some embodiments, the mechanical wave generator is indirectly attached to the solid surface, e.g., through another solid and/or liquid, and/or fluid phases, which are capable of propagating the mechanical wave. According to some embodiments, the mechanical wave generator is not attached to the solid surface.

According to some embodiments, the mechanical wave generator contacts the liquid solution. According to some embodiments, the mechanical wave generator does not contact the liquid solution.

According to some embodiments, the mechanical wave propagates through the liquid solution. According to some embodiments, the mechanical wave propagates through the solid surface.

According to some embodiments, the mechanical wave generator (or signal generator) is selected from: an acoustic actuator, a mechanical-wave actuator, an ultrasonic actuator, a piezoelectric actuator, a surface acoustic wave actuator, and a surface acoustic wave device.

It is understood, unless indicated otherwise, that the term “mechanical wave” is intended to also encompass an electro-mechanical wave. An example for a mechanical wave is mechanical wave 108.

According to some embodiments, the mechanical wave is an electro-mechanical wave. According to some embodiments, the mechanical wave is an electro-mechanical wave in a piezoelectric solid.

According to some embodiments, the mechanical wave includes a lamb wave, a shear wave, a flexural wave, a Love wave, and/or a surface or surface acoustic wave (SAW). The surface wave or the SAW may be, e.g., a Rayleigh wave, a leaky Rayleigh wave, a Scholte wave, or a Sezawa wave or equivalently Sezawa mode wave.

According to some embodiments, the mechanical wave is selected from: a surface acoustic wave (SAW), a lamb wave, a shear wave, a flexural wave, a Love wave, a surface wave and, and combinations thereof.

According to some embodiments, the surface wave or SAW includes a Rayleigh wave, a leaky Rayleigh wave, a Scholte wave, a Sezawa wave, and/or a Sezawa mode wave, and combinations thereof.

According to some embodiments, the mechanical wave includes a standing (i.e., stationary) wave, a traveling wave, or a combination thereof. According to some embodiments, the mechanical wave includes a standing wave. According to some embodiments, the mechanical wave includes a traveling wave. According to some embodiments, the mechanical wave is a standing wave. It is noted that a standing wave is more suitable for measuring high ion concentrations.

Ions in solution have relaxation times which are a function of ion size (through the ion diffusion coefficient) and the Debye length of the electrical double layer. This feature of the ions is used in the present invention to allow their identification, since when the EDL is excited by mechanical wave frequencies which correspond to the resonance, or the relaxation time, of the ions in the EDL, the voltages (i.e., electrical potential) leaked from the EDL peaks. A wave frequency “corresponding” to an ion relaxation time is a wave frequency that is close to the inverse of the ion relaxation time, or close to the ion relaxation frequency, which is the inverse of the ion relaxation time. It can also be said that the mechanical wave period is close to the ion relaxation time. The same or similar effect is also observed when the mechanical wave frequency is close to a multiplication by an integer of the inverse ion relaxation time (wave harmonics), or to half the inverse ion relaxation time (half harmonics).

According to some embodiments, the mechanical wave includes at least one wave having a frequency that is close to the inverse, or half the inverse, of a relaxation time of at least one of the ions in the EDL. According to some embodiments, the mechanical wave includes at least one period that is close to a relaxation time of at least one of the ions in the EDL.

According to some embodiments, the mechanical wave includes at least one wave having a frequency that is close to the inverse of a relaxation time of at least one of the ions in the EDL multiplied by an integer.

According to some embodiments, the mechanical wave includes at least one wave having a frequency that is close to a relaxation frequency or half a relaxation frequency, of at least one of the ions in the EDL. According to some embodiments, the mechanical wave includes at least one wave having a frequency that is close to a relaxation frequency of at least one of the ions in the EDL, multiplied by an integer.

According to some embodiments, the term “close to” as used herein, means less than about 20%, 15%, 10%, 7%, 5%, or 2% higher or lower than the item compared to. In some embodiments, the definition of the term “close to” applies to ion solutions including ion concentrations of less than about 100 mM (0.1M) in total, and may be higher for solutions of higher ion concentrations.

According to some embodiments, the frequency of the mechanical wave is about 1 KHz to about 1 GHz. According to some embodiments, the frequency of the mechanical wave is about 10 MHz to about 80 MHz. According to some embodiments, the frequency of the mechanical wave is about 20 MHz to about 50 MHz.

According to some embodiments, the mechanical wave has a substantially constant frequency.

According to some embodiments, the mechanical wave includes a band of waves, having a plurality of frequencies, i.e., the mechanical wave includes a plurality of waves including different frequencies. According to some embodiments, the mechanical wave includes a plurality of waves having a plurality of amplitudes.

According to some embodiments, the mechanical wave includes a single wave. According to some embodiments, the single wave has a single frequency and a single amplitude. According to some embodiments, the single wave includes more than one frequency and/or an amplitude that changes periodically in time.

The term “band” is used for indicating that the plurality of waves included in the band, and which include pluralities of frequencies and/or amplitudes, are generated simultaneously, or at the same time, by the wave generator.

The term “plurality” means at least two, such as 2, 3, 4, 5, 10, or more than 10, etc.

According to some embodiments, the plurality of frequencies is in a range of about 1 KHz-1 GHz, about 10 MHz-80 MHz, about 20 MHz-50 MHz, or any range falling inside these ranges.

It is appreciated that the mechanical wave generator may conduct a frequency sweep (also known as a frequency span, or frequency modulation), by modulating the frequency of the generated mechanical wave during the measurement, throughout a pre-defined range list of frequencies. In this case, measurements are taken for each frequency so that the mechanical wave frequency may be correlated with the resulting leaked voltage intensity.

The pre-defined range may correspond to any range within frequencies noted herein for the mechanical wave. The frequency sweep may sweep through consecutive frequencies or through non-consecutive frequencies, such a through a list of desired discrete frequencies. At each time during the sweep, the wave generator may generate a single wave, having a single frequency, or a plurality of waves, having a plurality of different frequencies.

Accordingly, in some embodiments, the mechanical wave generator includes a frequency modulator for temporally modulating the frequency of the mechanical wave throughout the measuring of the electrical field over time.

According to some embodiments, the mechanical wave has temporally changing frequencies.

Instead of changing the wave frequencies, it is also possible to temporally modify the wave displacement (strain) amplitude or equivalently temporally modify the wave intensity while keeping the wave frequencies constant.

Accordingly, in some embodiments, the mechanical wave generator includes an amplitude modulator for modulating the amplitude of the mechanical wave throughout the measuring of the electrical field over time. For example, a mechanical wave may be generated at a certain frequency (herein termed “carrier frequency”), followed by modulating the amplitude (i.e., intensity), such as the particle velocity amplitude or the displacement (strain) amplitude of the mechanical wave at a “modulation frequency”, which is the frequency at which the modulated amplitude is applied to the carrier wave. The resulting wave has fixed frequencies and a temporally changing displacement (or particle velocity) amplitude.

Accordingly, in some embodiments, the mechanical wave generator includes an amplitude modulator for temporally modulating the amplitude of the mechanical wave throughout the measuring of the electrical field over time.

According to some embodiments, the mechanical wave has temporally changing frequencies and/or amplitudes.

According to some embodiments, the mechanical wave results from amplitude modulation and includes temporally changing amplitudes.

According to some embodiments, the mechanical wave generator is configured to modulate the frequency of the mechanical wave in a range of about 1 KHz-1 GHz, 10 MHz 80 MHz, 20 MHz-50 MHz, or any range falling inside these ranges.

The working microelectrode, such as working electrode 110, is used for measuring the electric field leaked from the EDL throughout the experiment, for each wave frequency. It is important that the working electrode does not directly contact the solid surface, but that is it in close proximity to the solid surface, since if it is too far from the EDL, the electrical field leaked from the EDL is lost or hindered by the ions in the solution and no useful information can be retrieved.

According to some embodiments, the working electrode is selected from a conductive metal, alloy or layered conductive metal. According to some embodiments, the working electrode is coated by non-conductive electric insulation, such as plastic. According to some embodiments, the working electrode is uncoated. According to some embodiments, the working electrode is a silver-plated copper wire. According to some embodiments, the working electrode is coated by an outer shrouding for insulation.

According to some embodiments, the working electrode has a diameter of about 50 μm-2,000 μm. According to some embodiments, the working electrode has a diameter of about 50-1,000, 50-500, 200-2,000, or 200-1,000 μm.

According to some embodiments, the working electrode is further connected to an oscilloscope, such as oscilloscope 114. According to some embodiments, the working electrode is further connected to a differential voltage probe. According to some embodiments, the working electrode is further connected to a lock-in amplifier. A lock-in amplifier may be needed when the measured voltage signal is of similar or smaller magnitude compared to the voltage-noise in the measurement. Voltage-noise is generated by electronic equipment used for the measurement and/or by external sources that could be artificial and environmental, e.g., radio transmissions and the sun, respectively. The lock-in amplifier extracts the signal at a specified frequency from the overall signal captured and average it over many periods of the measured harmonic signal. It is noted that it is possible to imitate a lock-in amplifier hardware by measuring the voltage signal using an oscilloscope and then analyzing the resulting signal output of the oscilloscope using lock-in algorithms (written as a computer code). Voltage signal of a specified frequency may then be extracted from the overall signal captured, when the electronic voltage noise level is greater than or similar to the magnitude of the signal generated by the ions in the EDL.

The term “close proximity”, as used herein, with reference to the position of the working electrode with respect to the solid surface, is meant to define a position that is sufficiently close to the surface in order to measure the EDL emissions without interference from ions in the solution, while not actually touching the surface. Generally, the working electrode is supposed to be in the solution and outside the EDL, and within about one wavelength of the mechanical wave away from the solid surface. Accordingly, in some embodiments, the term “close proximity” is less than a wavelength of the mechanical wave in the solid surface. For example, in the present application, a 20 MHz mechanical wave has been used, having a wavelength of about 200 microns.

The thickness of the EDL is typically about 0.5 nm to about 0.5 micron.

Accordingly, in some embodiments, the term “close proximity” means less than about 200, 150, 100, 50, or 40 microns from the solid surface. In some embodiments, the term “close proximity” means less than about 40 microns from the solid surface. In some embodiments, the term “close proximity” means about 40 microns from the solid surface.

According to some embodiments, the working microelectrode is positioned in the solution at a distance of less than about a wavelength of the mechanical wave from the solid surface, without contacting the solid surface. According to some embodiments, the working electrode is positioned in the solution between a few microns and a few tens of microns away from the solid surface, e.g., about 1-200, 1-100, 1-50, 5-200, 5-100, 5-50, 10-200, 10-100, 10-50, 20-200, 20-100, 20-200, 30-100, 30-70, or 30-50 microns from the solid surface, or less than about 200, 100, 70, 50, or 40 microns from the solid surface. According to some embodiments, the working electrode is positioned in the solution about 40 microns from the solid surface.

According to some embodiments, the working microelectrode is configured to move (e.g., closer to or further away from the solid surface) such that its position may be adjusted as needed.

According to some embodiments, the system further includes a reference electrode, such as reference electrode 112. According to some embodiments, the reference electrode is positioned in the solution at a distance of more than about a wavelength of the mechanical wave from the solid surface, preferably at least 10 wavelengths away from the solid surface. The reference electrode should be farther away from the solid surface than the working electrode, far enough from the surface so as to detect mainly background potential and not specifically the potential released by ions in the EDL. The reference electrode may be placed in the solution a few millimeters away from the solid surface, e.g., about 0.5-10, 0.5-5, 1-10, 1-7, 1-5 mm away from the solid surface, or at least about 1, 2, 3, 4, 5, or 10 mm away from the solid surface.

According to some embodiments, the reference electrode is connected to an external ground potential.

According to some embodiments, the reference electrode is selected from a conductive metal, alloy or layered conductive metal. According to some embodiments, the reference electrode is coated by non-conductive electric insulation, such as plastic. According to some embodiments, the reference electrode is uncoated. According to some embodiments, the reference electrode is a silver-plated wire. According to some embodiments, the reference electrode is coated by an outer shrouding for insulation.

It is appreciated that although the elements of the device are described herein according to certain positions (such as positioning of the electrodes) which are required for the device to properly function, the intention is to describe the device when in action. Accordingly, the device may be in a different configuration when not in active, and configured such that when in action, the elements are positioned as described herein.

According to some embodiments, the system further includes a processor which includes a computation module.

It is appreciated that although the processor may be a physical part of the system, is not necessarily a physical part of the system and may be an external processor, such as a computer, a server, or a smartphone etc. The processor may receive data, such as the potential/voltage spectrum measurements from the system (e.g. from the electrodes) by wire communication or by wireless communication, and remotely analyzes the data.

In some embodiments, the processor is communicatively associated with the system. In some embodiments, the processor is physically associated with the system. In some embodiments, the processor is associated with the system at least one wire. In some embodiments, the processor is a physical part of the system.

In some embodiments, the system further includes a wireless or a communication unit.

According to some embodiments, the computation module is configured to analyze the electrical potential or voltage spectrum measured over time and to provide an identify and/or a concentration of the one or more ions.

According to some embodiments, the computation module is configured to analyze the electrical field by identifying at least one peak amplitude or intensity of the voltage spectrum induced by a certain mechanical wave frequency, and identifying the one or more ions based on the ions having a relaxation frequency or half the relaxation frequency (alternatively, inverse or half inverse relaxation time), or a relaxation frequency (alternatively, inverse relaxation time) multiplied by an integer, that is close to the certain mechanical wave frequency which induced a peak in the voltage spectrum. It is noted that “close to” is defined hereinabove.

According to some embodiments, the computation module is configured to analyze the electrical field by identifying a series of peak amplitudes or intensities of the electrical field voltage spectrum, including a main peak at a wave frequency corresponding to the leading ion resonance harmonic (which causes the highest path length of the ion vibration) and smaller peaks corresponding to secondary harmonics (corresponding to shorter path lengths), which together form a fingerprint of the ionic mixture and concentration of the one or more ions.

As stated above, the term “relaxation frequency” is the inverse of “relaxation time”.

According to some embodiments, providing the concentration of the one or more ions is further based on the intensity of the peak related to the relaxation frequency of the ions.

According to some embodiments, the computing module may be in communication with a mobile/portable device.

According to some embodiments, the mobile/portable device includes one or more of: a smartphone, a laptop, a tablet, and a wearable device.

According to some embodiments, the system is configured to assess quality of water in public, domestic, and/or residential facilities.

According to some embodiments, the system is devoid of ion sensitive barriers.

According to some embodiments, the system is configured to identify the one or more ions and concentrations thereof simultaneously.

According to some embodiments, the system is further configured to compute zeta potential of the EDL based on the measured potential.

### Methods for Identifying Ions in Solution

According to some embodiments, there is disclosed herein a method for identifying one or more ions in a liquid solution, the method including:
a) contacting the solution with a solid surface;
b) generating, by a mechanical wave generator configured to be in contact with the solid surface and/or with the liquid solution, a mechanical wave including at least one frequency and at least one amplitude which propagates and excites ions in an EDL formed at an interface between the solid surface and the solution, thereby resulting in the EDL emanating an electrical field away therefrom;
c) measuring, by a working microelectrode configured to be positioned in close proximity to the solid surface without contacting it, for each frequency and/or amplitude in the mechanical wave, a voltage of the electrical field emanated from the EDL over time; and
d) analyzing, by a computation module in a processor, a spectrum of the voltage measured for frequencies and/or amplitudes included in the mechanical wave to identify the one or more ions and concentrations thereof in the solution.

In some embodiments, the voltage spectrum includes amplitude and phase indicative of the identity and the concentration of the one or more ions in the liquid solution.

Definitions and embodiments mentioned above and which may be relevant to the method embodiments also apply here, and vice versa. Some particularly relevant embodiments may be pointed out or explicitly repeated.

For terms used herein, unless stated otherwise, their definition and embodiments are intended to be the same as above (mutatis mutandis).

The mechanical wave generated may be of a single frequency, a band of a plurality of frequencies and/or amplitudes, or of a temporally changing frequency and/or amplitude, as discussed above. The wave excites ions in the ˜nanometer thick electrical double layer next to the solid surface that may be piezoelectric or non-piezoelectric and may be naturally or artificially electrically charged. The excited electrical double layer leaks an electrical field away from the electrical double layer, and the electrical field (or voltage spectrum) is measured by a working electrode positioned between a few microns and a few tens of microns away from the solid surface (not touching the surface). The measurement is typically against a reference electrode in the liquid, positioned approximately a millimeter or several millimeters away from the solid surface, or against the ground potential of a metallic body or of the building electrical system. As also explained above, peaks measured in the voltage leakage spectrum, which appear at certain frequencies of the mechanical wave, indicate that the frequency eliciting the peaks corresponds to a relaxation time of certain ions in the solution. The intensity of the peaks corresponds, inter alia, to the total concentration of the ions corresponding to the peaks. The correlation between the relaxation times of the ions in the liquid solution and the frequencies eliciting measured peak voltage leaks are described in more detail above.

Contacting the liquid solution with the solid surface is intended to generate an EDL, for which parameters are measured, as described above. The contacting may be in any way which achieves this goal.

According to some embodiments, contacting the liquid solution with the solid surface includes placing a drop of the liquid solution on the solid surface.

According to some embodiments, contacting the liquid solution with the solid surface includes at least partially immersing the solid surface in the liquid solution.

According to some embodiments, the method further includes adjusting the position of the working electrode in the solution (e.g., closer to the solid surface, or further away therefrom).

According to some embodiments, the working electrode is positioned in the solution at a distance of less than about a wavelength of the mechanical wave from the solid surface and without contacting the solid surface. According to some embodiments, the working electrode is positioned in the solution between a few microns and a few tens of microns away from the solid surface, e.g., about 1-200, 1-100, 1-50, 5-200, 5-100, 5-50, 10-200, 10-100, 10-50, 20-200, 20-100, 20-50, 30-200, 30-100, 30-70, or 30-50 microns from the solid surface, or less than about 200, 100, 70, 50, or 40 microns from the solid surface. According to some embodiments, the working electrode is positioned in the solution about 40 microns from the solid surface.

According to some embodiments, the method further includes positioning a reference electrode at a distance of more than about a wavelength of the mechanical wave from the solid surface, preferably at least 10 wavelengths away from the solid surface. According to some embodiments, the method further includes positioning a reference electrode further away from the solid surface than the working electrode, and at a distance of a few millimeters away from the solid surface, e.g., about 0.5-10, 0.5-5, 1-10, 1-7, 1-5 mm away from the solid surface, or at least about 1, 2, 3, 4, 5, or 10 mm away from the solid surface. According to some embodiments, the method includes connecting the reference electrode to an external ground potential.

According to some embodiments, generating a mechanical wave in step (b) includes generating a plurality of waves including a plurality of different frequencies and/or a plurality of different amplitudes.

The plurality of waves may be generated simultaneously (such as in a band of frequencies), or sequentially, by sweeping or spanning over different frequencies, as further explained above.

According to some embodiments, generating a mechanical wave in step (b) includes temporally sweeping through a range or a plurality of frequencies, and for each frequency measuring in step (c) the voltage leaked from the EDL. Such a frequency sweep (or span, or modulation) may be conducted by a frequency modulator part of the mechanical wave generator, as explained above.

According to some embodiments, generating a mechanical wave in step (b) includes temporally modulating the mechanical wave amplitude, and for each amplitude measuring in step (c) the voltage leaked from the EDL. Such an amplitude modulation may be conducted by a amplitude modulator part of the mechanical wave generator, as explained above.

When a frequency sweep and/or amplitude modulation is performed at step (b), the corresponding voltage leaked from the EDL for each frequency and/or amplitude is measured (step (c)), and the process is repeated for all desired frequencies or amplitudes. Step (d) is conducted after the frequency sweep or the amplitude modulation is done, and a voltage spectrum, including the voltages obtained in response to the different frequences (different frequencies obtained by the amplitude modulation), is analyzed.

Alternatively, instead of spanning wave frequencies or modulating wave amplitudes, it is also possible to change the concentration of ions in the solution, e.g., by adding increasing amount of water. Changing the concentration affects the Debye length of the EDL, thereby changing the relaxation time of the ions in the solution.

Accordingly, in some embodiments, generating a mechanical wave in step (b) includes temporally modulating at least one concentration of the one or more ions. Similar to the above cases of wave modulation, voltage is measured in step (c) for each concentration, and the analysis in step (d) is done after voltage corresponding to all concentrations have been measured.

According to some embodiments, at least one of the one or more ion species is not known in advance of the method. According to some embodiments, the concentration of at least one of the one or more ion species is not known in advance of the method.

According to some embodiments, identifying the one or more ions and concentrations thereof includes applying computational algorithms.

According to some embodiments, analyzing the voltage spectrum in step (d) includes identifying at least one peak intensity of the voltage spectrum that is induced by a certain wave frequency, and identifying the one or more ions based on the ion having a relaxation frequency or half relaxation frequency (alternatively, inverse or half inverse relaxation time), or a relaxation frequency (alternatively, inverse relaxation time) multiplied by an integer, that is close to the certain frequency. It is noted that the term “close to” is defined hereinabove.

According to some embodiments, analyzing the electrical field in step (d) includes identifying a series of peak amplitudes or intensities of the electrical field voltage spectrum, including a main peak at a wave frequency corresponding to the leading ion resonance harmonic (which causes the highest path length of the ion vibration) and smaller peaks corresponding to secondary harmonics (corresponding to shorter path lengths), which together form a fingerprint of the ionic mixture and concentration of the one or more ion.

According to some embodiments, the analyzing includes applying a Fourier transform algorithm, such as a Fast Fourier Transform (FFT) or a Discrete Fourier Transform (DFT), and/or lock-in amplification algorithms.

For various reasons, such as the solution composition and concentration, the Debye length of the EDL formed in the liquid solution may be unknown. Since the relaxation time of an ion is affected by the Debye length, this means that the relaxation times of the ions in the solution are also unknown. To overcome this problem, the system may be calibrated by using known ions at known concentrations, which facilitate correctly calculating ion relaxation times.

As a non-limiting example, natural water typically contains sodium and chlorine, two elements abundant in our planet crust. According to some embodiments, by identifying the peaks of the two ions whose difference in relaxation time is well known, the relaxation times of the rest of ions may be identified and thus identify their sizes and hence their species. Once the ion species are known, the Debye length of the electrical double layer may be deduced and hence the concentration of the water or electrolyte solution specimen may be identified.

According to some embodiments, the method further includes, prior to generating a mechanical wave in step (b), a step of adding at least one known ion at a known amount to the liquid solution.

When known ions are added to the liquid solution, the known resonance frequencies of the known ions may be used to calculate the Debye length, as explained herein, which may then be used for finding unknown ions in the solution.

Additionally, a control solution, such as deionized water optionally including at least one known ion at a known concentration, may be used for calibrating, in order to subtract background voltage-noise.

Accordingly, in some embodiments, the method further includes performing steps (a)-(c) on at least one control solution including deionized water and/or a solution of at least one known ion at a known concentration, and the analyzing in step (d) includes adjusting the voltage spectrum obtained for the liquid solution based on the voltage spectrum obtained for the control solution.

According to some embodiments, the at least one control solution is several control solutions having varying concentrations of the at least one known ion or salt.

According to some embodiments, the control solution differs from the liquid solution only by including known ions or salts at known concentrations. According to some embodiments, the control solution includes common water ions.

According to some embodiments, the voltage spectrum measured from ion solution is adjusted by subtracting or dividing the voltage spectrum obtained for the control solution by the voltage spectrum obtained for the liquid solution.

According to some embodiments, the adjusting is performed by dividing the voltage spectrum obtained for the liquid solution by the voltage spectrum obtained for the control solution.

According to some embodiments, the at least one known ion includes sodium and/or chlorine ions. According to some embodiments, the at least one known ion is part of a known salt. According to some embodiments, the at least one known ion is part of a known salt selected from NaCl, KCl, NaNO₃, and MgSO₄.

According to some embodiments, the liquid solution is selected from fresh water, saline water, tap water, pool water, water in pipes, sea water, ocean water, river water, reservoir water, waste water, sewage water, biological fluids, bodily fluids, water used for industrial processes/facilities, or any combination thereof.

According to some embodiments, zeta potential is further computed based on the measured voltage.

### Theoretical Basis for the Invention

In the followings paragraphs, the theoretical basis for SAW excitation of an EDL and calculation methods used in the present invention, in some embodiments, are explained.

Reference is made to FIG. 1A, which presents an illustration of boundary layer flow streamlines, φ, in an electrolyte solution of positive and negative ions (+, −) next to a charge solid, releasing an electrical field, ψ, to the solution. FIG. 1B presents a theory for temporal (t) variations of the measured voltage in the presence and absence of an EDL, ψ₁ and ψₐᵢᵣ, respectively, following an application of a voltage signal, ψᵢ, to the piezoelectric actuator, and FIGS. 1C-1D show tᵣ(m⁺) variations of the measured voltage amplitude, |ψ₁|, and phase, ΔΘ, for different tᵣ(m⁺)/tᵣ(m⁻) ratios and for β=ψₛ=ψᵢ=1, where tᵣ(m⁺) and tᵣ(m⁻) are the characteristic acoustic to EDL time ratios for positive and negative ion species (m⁺, m⁻), respectively, and where tᵣ≡tᵣ(m⁺)=tᵣ(m⁻) for same ion diffusion coefficients. In the inset we assume β=ψₛ=tᵣ(m⁺)=tᵣ(m⁻)=1 to show that the |ψ₁| maxima, marked with circles, migrate to greater tᵣ(m⁺) values when increasing |ψᵢ|.

Dubrovski et al. (Langmuir 2021, 37, 14679-14687) suggested an electrokinetic theory for an EDL emanating from a flat solid and undergoing MHz-frequency vibration by a Rayleigh SAW, forecasting that the SAW excited EDL should leak an electrical signal modulated by EDL properties. Here, the theory is altered to include intrinsic complexities that appear in measurement, such as different diffusion coefficients for different ion species, SAW attenuation under a drop of electrolyte solution, and the finite size of the electrode in experiment; the electrode spatially averages over variations in the electrical leakage along its surface area.

These insights are introduced by solving the Navier Stokes and continuity equations for a solenoidal flow field in the solution, the Nernst-Planck equations for transport of univalent positive and negative ion concentrations, c⁺ and c⁻, that are associated with the diffusion coefficients D(m⁺) and D(m⁻) respectively, and the Poisson equation for the electrical field distribution, assuming the Cartesian coordinates and system geometry in FIG. 1(a). Flow and ion dynamics appear in an electrolyte solution neighboring an attenuating Rayleigh type SAW in a flat solid substrate; the piezoelectric solid further supports a voltage wave that accompanies the mechanical wave, i.e., the solid displacement.

The system of equations is rendered dimensionless using the scaling transformations for time, coordinates along and transverse the flat solid surface at rest, stream function (representing the velocity field in the solution), electrical potential, SAW attenuation length, and ion positive and negative concentrations in the electrolyte solution, t→t/ω, x→x/κ, y→σy, φ→δ Uφ, ψ→(k_{B}T/e) ψ, α→κ α, c⁺→χ c⁺, c⁻→χ c⁻ respectively, where κ, U, and χ are the wavenumber and deformation velocity amplitude of the SAW at the solid surface and the concentration of the univalent electrolyte (salt) in the bulk of the solution, respectively. The ion concentration and electrical field are governed by the Nernst-Planck and Poisson equations,

$${c}_{t}^{+} + {St}^{- 1} ( {ϕ}_{y} {c}_{x}^{+} - {ϕ}_{x} {c}_{y}^{+} ) = {t}_{r} ( {m}^{+} ) ( {c}_{yy}^{+} + {ψ}_{y} {c}_{y}^{+} + {ψ}_{yy} {c}_{1} ) , \qquad ( 1 )$$

$${c}_{t}^{-} + {St}^{- 1} ( {ϕ}_{y} {c}_{x}^{-} - {ϕ}_{x} {c}_{y}^{-} ) = - {t}_{r} ( {m}^{-} ) ( {c}_{yy}^{-} + {ψ}_{y} {c}_{y}^{-} + {ψ}_{yy} {c}_{2} ) , \qquad ( 2 )$$

$$2 {ψ}_{yy} = {c}^{+} - {c}^{-} , \qquad ( 3 )$$

where St≡ω/κU×σ/δ, Iᵣ≡δ/σ, and Ol≡(k_{B}Tχ)/(ηU/σ)×σ/κ give the characteristic ratio between viscous and inertial contributions to the flow (Strouhal number), between the SAW viscous penetration length and the Debye length, and between osmotic pressure and shear stress in the solution, respectively. Moreover, tᵣ(m⁺)≡D(m⁺)/σ¹ψ and tᵣ(m⁻)≡D(m⁻)/σ²ω give the ratio between the acoustic time scale and EDL relaxation times for the positive and negative ions (m⁺ and m⁻), respectively, and a shorthand notation was used for derivatives; for example, φ_{y}≡∂φ/∂y and φ_{κ}≡−∂φ/∂x represent flow velocity along and transverse to the flat solid surface at rest, respectively.

The leading order contribution to the electrical field leakage off the EDL is dependent on the leading order flow field in the electrolyte solution. This is a product of the Navier Stokes and continuity equations and is powered by the normal deformation of the solid surface by the SAW²⁵. It is given by φ₀=(κδ)⁻¹(1+α²)⁻¹(sin(t−x)+α cos(t−x))e^{-αx}, where it is assumed that the flow is near the solid surface, i.e., y<<1/κσ, and where the exponential attenuation of the SAW is accounted for with an attenuation length α⁻¹ under the drop at x>0. Thus, the upstream three phase contact line of the drop is positioned between the solid, air, and solution at the origin (x=0), where the SAW first encounters the liquid and commences attenuation. The stream function solution, φ₀, is a mere leading order product of mass conservation to result from the normal deformation velocity of the SAW at the solid surface (κδ)⁻¹ cos(t−x)e^{-αx}. In a piezoelectric substrate, the mechanical deformation is accompanied by a spatiotemporal voltage variation that takes the form ψᵢ cos(t−x)e^{-αx} at the solid surface, where ψᵢ is the voltage amplitude.

Assuming that the Stern layer in the EDL behaves like a linear capacitor for simplicity gives the boundary condition for the electrical field (ψₛ−ψ)+βψₙ=0 at y→h(x), where β≡ϵ₀ϵᵣ/σCS is the scaled inverse capacitance, n is the normal to the solid surface, and ψₙ is the normal derivative of the electrical potential. In addition, to account for a piezoelectric substrate, it is assume that the electrical potential in the solid surface at y=0 is ψ≈ψₛ+ψᵢ cos(t−x)e^{-αx}, where ψₛ and ψᵢe^{-αx} are constants and transient contributions to the electrical potential, respectively. The former is to leading order a product of the solid surface chemistry, and the latter is associated with the attenuating voltage that accompanies the attenuating mechanical wave in the piezoelectric solid under the drop. Complications such as permittivity variations and electroviscous effects are avoided. Far from the solid, a vanishing electrical potential is assumed, as well as that ion concentrations reach their bulk counterparts in the drop, i.e., ψ_{y}=ψ_{yy}=ψ_{yyy}=0 and that c⁺=c⁻=1 and c_{y}⁺=c⁻_{y}=0 as y→∞.

The physical insights that δk>>St⁻¹ and δk>>Ol in the 1 MHz to 1 GHz SAW frequency range, regardless of ionic strength and electrical potential, motivate the equation expansion methodology: we assume the small parameter ϵ≡St⁻¹≈Ol≈(κδ)²<<1 and represent the different variables using the asymptotic series

$$ϕ = {ϵ}^{- 1 / 2} {ϕ}_{0} + ⋯ , ψ = {ψ}_{0} + {ϵ}^{1 / 2} {\tilde{ψ}}_{1} + ⋯ ,$$

$${c}^{+} = {c}_{0}^{+} + {ϵ}^{1 / 2} {c}_{1}^{+} + ⋯ , {c}^{-} = {c}_{0}^{-} - {ϵ}^{1 / 2} {c}_{1}^{+} + ⋯$$

The gauge functions (powers of ϵ) in the series alongside assuming that ψₛ=O(1) and ψᵢ=O(ϵ^{1/2}) render the leading order electrical potential independent of SAW. The leading order solution of the electrical potential, ψ₀=ψₛ(1+β)⁻¹e^{-y}, satisfies the linear Poisson-Boltzmann equation for small electrical potential beyond the Stern layer. The ion concentrations satisfies the Boltzmann distribution

$${c}_{0}^{+} = {e}^{- ψ} , {c}_{0}^{-} = {e}^{ψ} .$$

The second order electrical potential, ψ⁻₁, is given from the leading order corrections to equations (1), (2), and (3) and is solved by assuming a solution of the form

$${\tilde{ψ}}_{1} = {\sum }_{j = 0}^{∞} {a}_{j} {e}^{- j y} {e}^{i ( t - x )} {e}^{- α x} ,$$

where i=\sqrt{−1} and aⱼ are constants, which are evaluated by a Gram-Schmidt process. The solution is terminated at j>2; the contribution of the following terms is approximately two orders of magnitude smaller. Moreover, unlike the leading order electrical potential, ψ₀, the solution to the second order electrical field contribution, \tilde{ψ}₁, contains a finite a₀ coefficient and does not vanish far from the solid surface—an electrical leakage off the EDL.

To compare the two dimensional theory with experiment, the limit of the solution for the electrical potential is obtained far from the solid surface—the electrical leakage—for weak SAW attenuation, α<<1. The limit is independent of α and is compatible with the experiment: The SAW wavenumber κ=10⁴π l/m scales the SAW attenuation coefficient; the dimensionless product is small, i.e., α≈1/10π<<1. Moreover, to account for the finite electrode size, this result is spatially averaged,

$${ψ}_{1} = ( 1 / l ) {\int }_{0}^{l} {\tilde{ψ}}_{1} ( y \to ∞ , α \to 0 ) dx ,$$

along the l=2.5π diameter (dimensional 2.5×10⁻⁴ m) of the circular electrode surface, which parallels the solid, while assuming the application of a voltage signal to the piezoelectric actuator to generate SAW, ψᵢ cos(t) at x=0.

FIG. 1B shows the voltage signal transmitted to the piezoelectric actuator (ψᵢ), the measured voltage signal in the absence of EDL, ψₐᵢᵣ, and the measured voltage signal in the presence of EDL, ψ₁, for different SAW to EDL characteristic time ratios, tᵣ≡tᵣ(m⁺)=tᵣ(m⁻) (assuming both ion species, m⁺ and m⁻, possess the same diffusion coefficient). Varying the ratio, tᵣ, signal variations in magnitude and phase are observed, which indicate an interaction between the EDL and SAW.

Concentrating on the magnitude and phase of the measured signal, ψ₁, the positive ion based time ratio (tᵣ(m⁺)) variations of the measured to transmitted signal amplitude ratio, |ψ₁|/|ψᵢ| are plotted in FIG. 1C. Results for different diffusion coefficient ratios of the positive and negative ions, tᵣ(m⁺)/tᵣ(m⁻), are shown, providing two insights: The measured voltage variation is not monotonous. It should obtain maxima near tᵣ(m⁺)=1 and tᵣ(m⁻)=1. At these points, the SAW disturbance of ions and their relaxation toward an equilibrium EDL are correlated in time. Ion displacement is at local maximum, which results in a local maximum in the electrical leakage off the EDL. When tᵣ(m⁺)≈tᵣ(m⁻)≈1, the two maxima, which correspond to the two ions, are close and essentially merge to one maximum. When tᵣ(m⁺)>tᵣ(m⁻), the formation of two separate maxima, one for each ion species, are observed. In the inset a similar plot is provided, assuming for simplicity that tᵣ(m⁺)≈tᵣ(m⁻)≈1, and show that the maxima of |ψ₁|/|ψᵢ| are obtained at increasingly larger values of tᵣ(m⁺) when the applied voltage level |ψᵢ| is increased.

In FIG. 1D, the phase difference between the signal phase in the presence and absence of EDL, ΔΘ, that correspond to the measured electrical leakage |ψ₁|ψᵢ| in FIG. 1C, was further plotted. The measured signal appears at the same phase in the presence and absence of the EDL for large ion strength, where the Debye length, σ, and hence the relaxation time of the EDL become small (tᵣ(m⁺), tᵣ(m⁻)>>1). Essentially, the ionic structure in the EDL adapts very fast to the SAW agitation to maintain a quasi-steady state. In the opposite case, at low ionic strength, where tᵣ(m⁺), tᵣ(m⁻)<<1, the agitation of the EDL by the SAW is such that the relaxation time of the EDL is large and the ions are almost freely displaced by the mechanical and electrical fields emitted by the SAW. Between these two limiting states a ΔΘ transition zone near tᵣ(m⁺)=1 and tᵣ(m⁻)=1 is observed. The transition slopes are monotonous or support a deflection point in the presence of one or two maxima in the measured voltage variation, respectively.

As used herein, the term “about” may be used to specify a value of a quantity or parameter (e.g. the length of an element) to within a continuous range of values in the neighborhood of (and including) a given (stated) value. According to some embodiments, “about” may specify the value of a parameter to be between 80% and 120% of the given value. For example, the statement “the length of the element is equal to about 1 m” is equivalent to the statement “the length of the element is between 0.8 m and 1.2 m”. According to some embodiments, “about” may specify the value of a parameter to be between 90% and 110% of the given value. According to some embodiments, “about” may specify the value of a parameter to be between 95% and 105% of the given value.

As used herein, according to some embodiments, the terms “substantially” and “about” may be interchangeable.

The following examples are presented in order to more fully illustrate some embodiments of the invention. They should in no way be construed, however, as limiting the broad scope of the invention. One skilled in the art can readily devise many variations and modifications of the principles disclosed herein without departing from the scope of the invention.

### Examples

### Experimental Setup

The experimental setup is comprised from a 20 MHz-frequency traveling SAW of 200 μm wavelength in a piezoelectric flat solid substrate on which a sessile drop of electrolyte solution is dispensed. Working and reference microelectrodes and, respectively (250 μm diameter) are positioned in the drop. The microelectrodes are made from a 30 AWG wire-wrap with an inner conductor of silver-plated copper wire having 0.25 mm diameter and an outer shrouding for insulation (KYNAR460, Pro-Power). The working electrode, designed for measuring the electrical leakage, ψ₁, is placed at a separation of 40 μm away from the solid surface to avoid the anticipated dispersion of the leakage at separations greater than the 200 μm SAW-wavelength. The reference electrode, which accounts for the electrical potential at the bulk of the drop, is placed at a separation of approximately 1 mm away from the solid. The electrodes are connected through an active differential voltage probe (RT-ZD02, Rohde & Schwarz, Germany) to an oscilloscope (HMO3004, Rohde & Schwarz, Germany) for obtaining frequency variation and to an RF lock-in amplifier (SR844, Stanford Research Systems, CA) for measuring the 20 MHz-frequency component of the electrical leakage, i.e., the linear (same frequency) response of the EDL to the exciting SAW. Moreover, sodium nitrate (NaNO₃), potassium chloride (KCl), sodium chloride (NaCl), and magnesium sulfate (MgSO₄), strong electrolytes of small ion pairs, are used separately for producing the electrolyte solutions, so that the EDLs are near ideal. Excluding potassium chloride, the ion pairs possess different diffusion coefficients.

### SAW Device

The SAW devices used (piezoelectric actuators) are comprised of about a 5 nm chrome/1000 nm aluminum interdigital transducer (IDT), consisting of 30 electrode pairs of 50 μm width and gaps (between electrodes) and an IDT aperture of 6 mm. The electrodes were patterned using a standard photolithography, on a 0.5 mm thick 128° Y-cut, X-propagating single crystal lithium niobate (LiNbO₃) piezoelectric substrate (Roditi International, England) to support a traveling 20 MHz-frequency Rayleigh type SAW. A signal generator (SMB100A, Rohde & Schwarz, Germany) is used to supply 20 MHz voltage signals to generate SAW in the LiNbO₃ substrate and a BNC T-connector (50Ω, Cinch Connectivity Solutions) is used to split the signal so that the same signal is simultaneously supplied to the lock-in amplifier reference signal port.

The SAW device is placed on a vertical translation stage (7VT40-13 Vertical Stage, Standa, Lithuania) while the differential voltage probe is held in position using a stand. To adjust the gap between the microelectrode and the SAW substrate, the position of the microelectrode is fixed and the height of the LiNbO₃ surface is varied with a vertical translation stage fitted with a micrometer of 13 mm a travel range and 1 μm resolution.

### Preparation of Electrolyte Solutions

The electrolyte solutions are prepared by dissolving ultrapure salts: NaNO₃ (Sodium nitrate, ACS reagent, ≥99.0%, Sigma-Aldrich, Germany), KCl (Potassium Chloride, ACS reagent, 99.0%, Sigma-Aldrich, Germany), NaCl (Sodium Chloride, ACS reagent, ≥99.0%, variation Chemical MFG CORP, CA), and MgSO₄(Magnesium Sulphate, Anhydrous Powder Reagent, ≥99.0%, variation Chemical MFG CORP, CA) in HPLC water.

### Results and Discussion

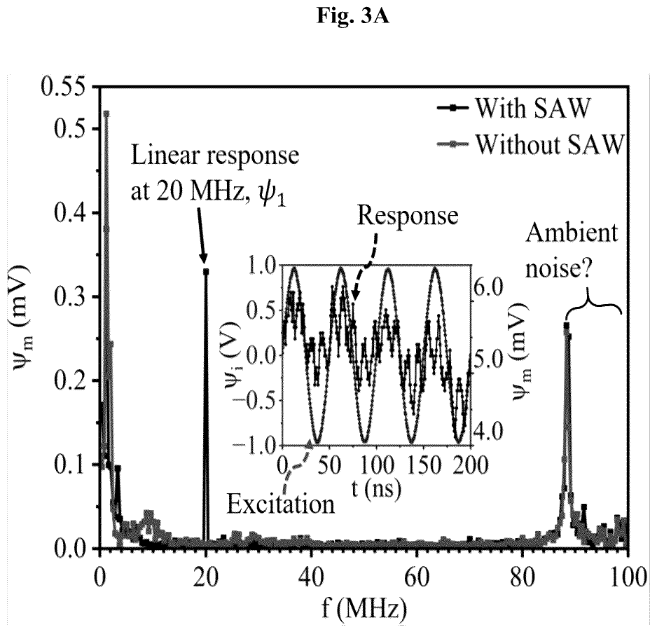

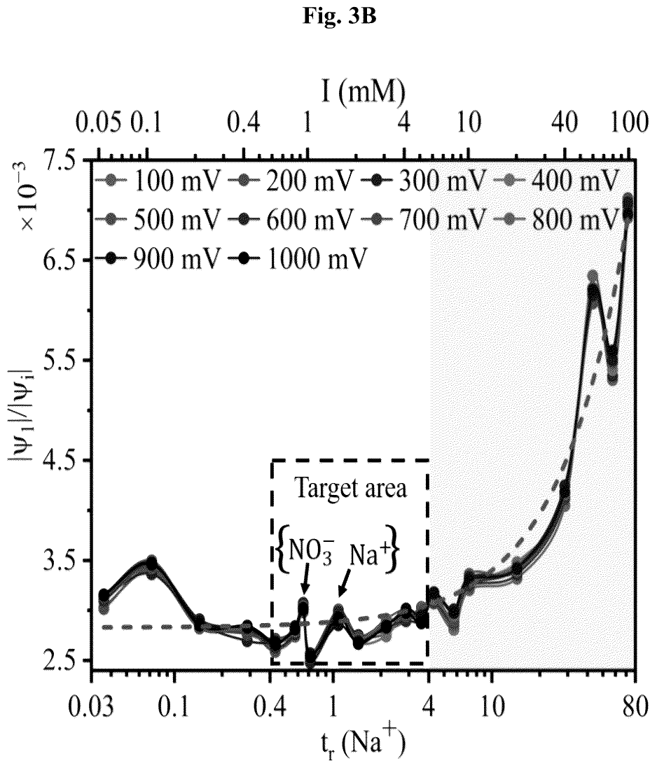

FIG. 3A depicts a characteristic Fast Fourier Transform (FFT) of the total electrical leakage, ψₘ, in a drop of 1.5×10⁻³ Molar NaNO₃ solution in the presence (black line) and absence (red line) of SAW. The 20 MHz FFT peak gives the linear response, ψ₁, of the EDL to SAW excitation. Peaks at frequencies below 10 MHz and between 80-100 MHz are likely ambient noise: These peaks are mostly present in the signal in the presence and absence of the SAW. In the inset, the original voltage signal, ψᵢ, is shown, applied to the piezoelectric actuator and the measured total EDL response, ψₘ. The measured electrical leakage, ψₘ, is two to three orders of magnitude lower than the excitation voltage, ψᵢ. This result is compatible with the theory given here, which depicts a corresponding magnitude ratio of ϵ^{1/2}≈δκ≈10⁻³π.

Next the 20 MHz component of the electrical leakage, ψ₁, is focused on.

FIG. 3B, shows ionic strength (I) spectra of electrical leakage amplitude, |ψ₁|, for different levels of the applied electrical signal amplitude |ψᵢ|. In the same figure, variations of the same parameters with the ratio, tᵣ(Nₐ⁺), between the acoustic time (ω⁻¹) and the sodium ion (Na+) relaxation time in the EDL (the characteristic time at which the ion attains equilibrium distribution) are further shown. In the figure, the electrical leakage increases linearly in magnitude with increasing ionic strength, apparent in particular in the shaded part of the figure at tᵣ(Nₐ⁺)>4. The dashed red line is a least square fit of the linear function |ω₁|≈a+b×I to the measured result. It connects the increase in |ψ₁| to an increase in solution conductivity. The latter is linearly proportional to the ionic strength, I, at law ion concentration according with the Ostwald dilution law and the empirical Kohlrausch law.

Next, the non-monotonic electrical leakage near tᵣ(m⁺)=1 and tᵣ(m⁻)=1, illustrated in the dashed box in the figure, is focused on.

### Ion Resonance and LC Circuit Analogy

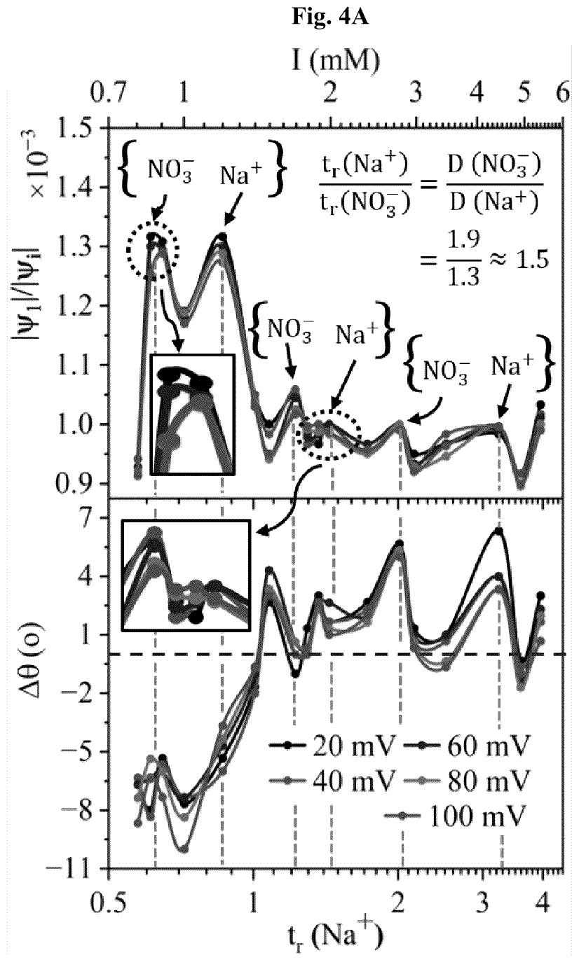

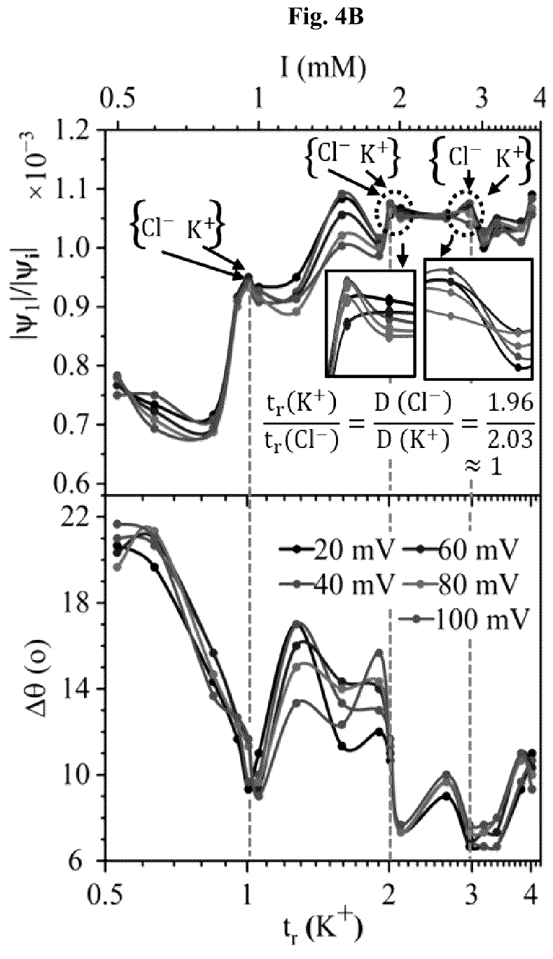

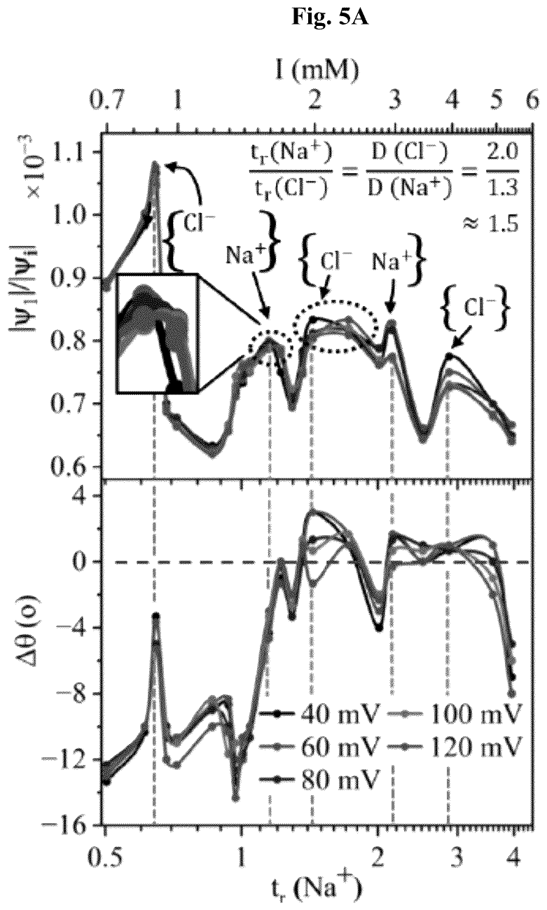

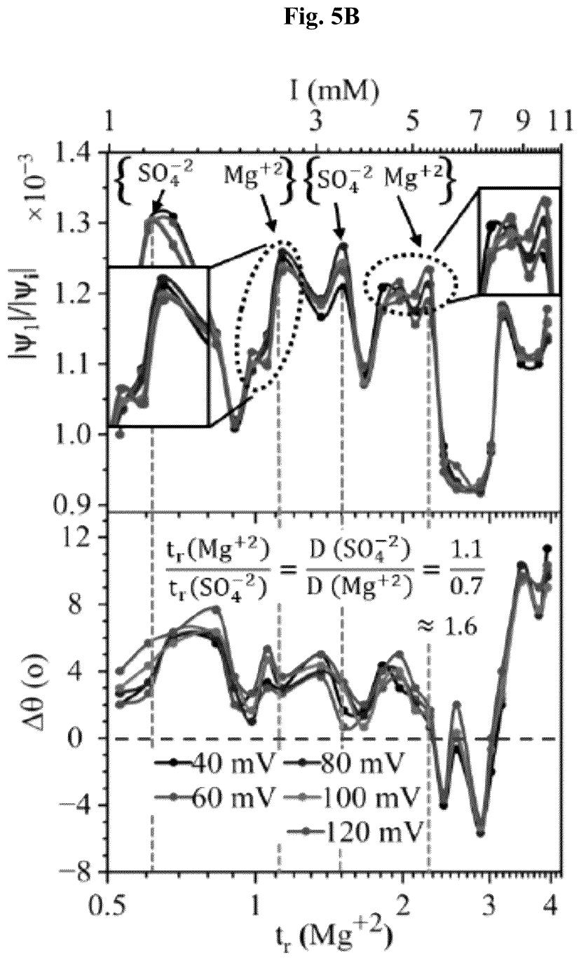

FIG. 4 & FIG. 5 demonstrate ionic strength (I) variations of electrical leakage magnitude, |ψ₁|, and phase difference, ΔΘ. As before, in the same figures, variations of the same parameters with the ratio, tᵣ(m⁺), between the acoustic time (ω⁻¹) and the relaxation time in the EDL of the positive ions in the different electrolyte solutions (m⁺) are further shown. Similarly to theory, |ψ₁| is maximized near tᵣ(m⁺)=1 and tᵣ(m⁻)=1. Since tᵣ(m⁻)/tᵣ(m⁺)=D(m⁺)/D(m⁻), The different sub-figures in FIG. 4 and FIG. 5 show, a |ψ₁| maximum near tᵣ(m⁺)=1 and a second maximum shifted on the tᵣ(m⁺) axis in a manner proportional to the diffusion coefficient ratio, D(m⁺)/D(m⁻), i.e., near tᵣ(m⁻)=tᵣ(m⁺)×[D(m⁺)/D(m⁻)]=1.

Results for sodium nitrate (NaNO₃) are shown in FIG. 4A, for potassium chloride (KCl) in FIG. 4B, for sodium chloride (NaCl) in FIG. 5A, and for magnesium sulfate (MgSO₄) in FIG. 5B. The salts break in water to ion pairs of different diffusing coefficients, yielding a different acoustic to EDL relaxation time ratio per ion:

$$\begin{matrix}{t}_{r} ( {Na}^{+} ) / {t}_{r} ( {NO}_{3}^{-} ) = D ( {NO}_{3}^{-} ) / D ( {Na}^{+} ) \approx 1.5 , {t}_{r} ( {Na}^{+} ) / {t}_{r} ( {Cl}^{-} ) \\ = D ( {Cl}^{-} ) / D ( {Na}^{+} ) \approx 1.5 ,\end{matrix} and$$

$${t}_{r} ( {Mg}^{+ 2} ) / {t}_{r} ( {SO}_{4}^{- 2} ) = D ( {SO}_{4}^{- 2} ) / D ( {Mg}^{+ 2} ) \approx 1.6 .$$

The ion mixtures give pairs of |ψ₁| maxima. FIG. 4B further shows measurement results for potassium chloride (KCl) that breaks in water to ion pairs of similar diffusing coefficients: tᵣ(K⁺)/tᵣ(Cl⁻)≈1. The ion mixture gives a small maximum for both ions. It is likely that conducting a similar measurement at greater ionic strength (I) resolution, sufficient to differentiating between the similar but not identical diffusion coefficients of the two ion species, will reveal a pair of maxima in this case as well. Moreover, not appearing in the linear theory, higher harmonics of the above mentioned “zero order” |ψ₁| maxima at near-integer multiplications of tᵣ(m⁺) and tᵣ(m⁻) are further observed.

The phase difference ΔΘ is the difference between the phase measured in the drop and the one measured in air (in the absence of a drop), keeping the rest of the parameters and especially the position of microelectrodes the same. The absolute magnitude of ΔΘ are near minima values at ionic strength corresponding to electrical leakage magnitude (|ψ₁|) maxima. The measurements depict an ionic strength variation of |ψ₁| and minimization of ΔΘ near |ψ₁| maxima distinctive of the mixture of ions in the electrolyte.

The local measured |ψ₁| maxima are due to a local decrease in the EDL electrical field screening efficiency, which is accompanied by a minimization of ΔΘ. These phenomena are reminiscent of electrical impedance decrease and signal phase minimization in an LC circuit near resonance. Equivalent electrical circuits methodology is used for realizing the dynamics of super capacitors—an energy storage systems relying on EDLs—albeit such systems comprise additional circuit components in addition to EDLs.

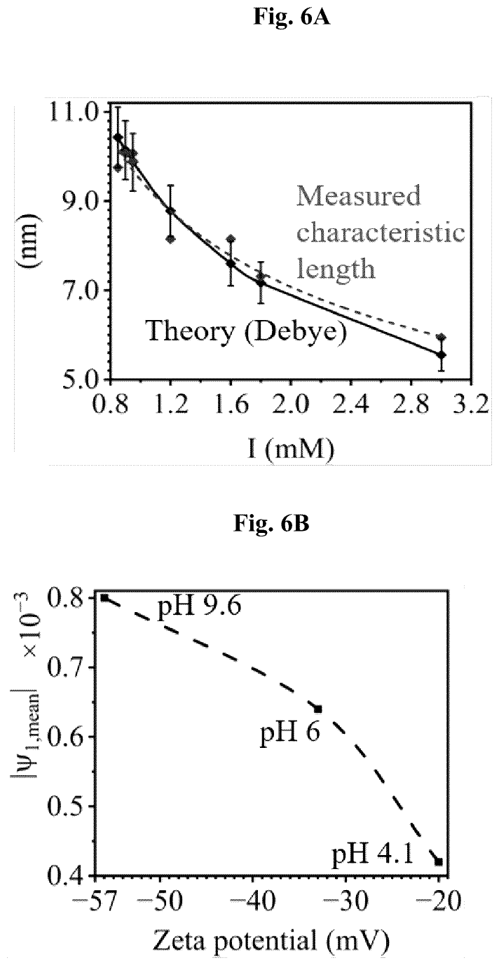

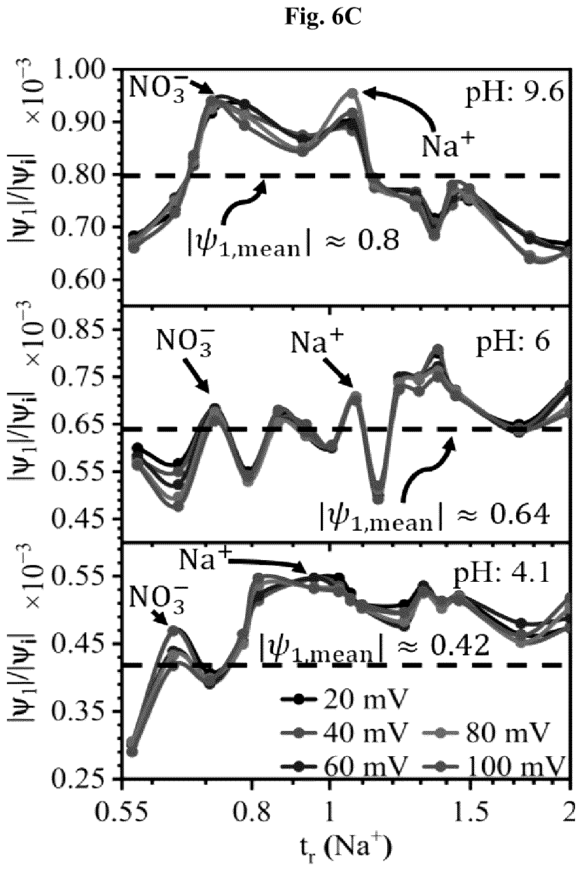

Realizing the dynamics of ions in the SAW excited EDL requires an insight into the path-length for ion motion, [symbol] . The Debye length, σ, is replaced with [symbol] in the definition for tᵣ(m⁺) and tᵣ(m⁻). The values of tᵣ(m⁺) and tᵣ(m⁻) that correspond to the ‘zero order’ |ψ₁| ‘resonance’ peaks in the different experiments give the [symbol] values in FIG. 6A. Following the previous analysis, one should expect that these values of [symbol] are comparable with the corresponding a values within the experimental uncertainty of ±10% in the ionic strength I. A byproduct of this analysis is that one may obtain the concentration of an electrolyte solution by evaluating a using the assertion that tᵣ(m⁺)=1 or tᵣ(m⁻)=1 at the I levels that correspond to the ‘zero order’ |ψ₁| peaks. Further considering harmonics of |ψ₁|, where tᵣ(m⁺)≈n, tᵣ(m⁻)≈n and n=1, 2, 3, . . . , the corresponding path-length for ion motion per an acoustic period is [symbol] ≈σ/\sqrt{n}. Hence the path-length for ion motion per an acoustic period decreases like 1/\sqrt{n} at harmonic n of |ψ₁|.

Lastly, the acidity of the drop—the level of pH—is varied to study the connection between the surface potential, ψₛ, which is pH dependent, and the level of electrical leakage, |ψ₁|.

In FIG. 6B independently measured pH variations of the zeta potential at the solid surface (in the absence of SAW) are plotted against the arithmetic mean of the electrical leakage magnitude, |ψ_{1,mean}|, at similar pH levels (pH=4.1, 6.0, and 9.6) in sodium nitrate solution, which is provided in FIG. 6C. The connection between the averaged magnitude of |ψ_{1,mean}| and the measured zeta potential is monotonic. Assuming that ψₛ is the surface potential of the solid—a product of the solid surface chemistry—and that it is unhindered by the voltage signal in the piezoelectric solid, which supports SAW, the measured zeta potential is proportional to ψₛ, indicating a monotonic connection between ψₛ and |ψ_{1,mean}| in agreement with the theory.

The path-length of ion vibration is comparable to the Debye length at the leading ion resonance harmonic, where an ion relaxation time in the EDL is comparable with the acoustic period. Secondary harmonics support shorter path lengths of the spatially vibrating ion and, many times, smaller peaks in the spectrum of the electrical leakage. The ion-specific electrical leakage spectrum translates to “fingerprints” of the ionic mixture and concentration in the electrolyte.

The corresponding phase difference magnitude between the phases of the electrical field leakage in the presence and absence of an EDL is locally minimized near the different harmonics of ion resonance, where the electrical leakage peaks. The frequency at which the electrical leakage peaks quantify the rate whereby different ions respond to dynamic changes in electrical potential and convective flow and thus gives the intrinsic, ion specific, relaxation time for EDL charge and discharge, e.g., a limiting ion-specific EDL response time in supercapacitors and dynamic electrophoretic and electrowetting systems.

1-40. (canceled)

41. A system for identifying one or more ions in a liquid solution, the system comprising:
a solid surface configured to be in contact with the liquid solution;
a mechanical wave generator configured to be in contact with the solid surface and/or with the liquid solution and to generate a mechanical wave, the mechanical wave being configured to propagate and excite ions in an electrical double layer (EDL) formed at an interface between the solid surface and the liquid solution, thereby causing the ions of the EDL to vibrate and to emanate an electrical field away therefrom; and
a working microelectrode configured to be positioned in the liquid solution in close proximity to the solid surface without contacting it, and to measure electrical potential resulting from the electrical field emanating from the EDL over time.

42. The system of claim 41, wherein the electrical potential measured over time yields a voltage spectrum comprising amplitude and phase which are indicative of an identity and a concentration of the one or more ions in the liquid solution

43. The system of claim 41, wherein the solid surface is a piezoelectric solid surface.

44. The system of claim 41, wherein the mechanical wave is selected from the group consisting of a surface wave or surface acoustic wave (SAW), a lamb wave, a shear wave, a flexural wave, a Love wave, and combinations thereof.

45. The system of claim 44, wherein the surface wave or the SAW is selected from the group consisting of a Rayleigh wave, a leaky Rayleigh wave, a Scholte wave, a Sezawa wave, and combinations thereof.

46. The system of claim 41, wherein the mechanical wave has a frequency of about 1 KHz to about 1 GHz.

47. The system of claim 41, wherein the mechanical wave comprises at least one wave having a frequency which is close to a relaxation frequency or to half the relaxation frequency, of the one or more ions, or close to the relaxation frequency multiplied by an integer.

48. The system of claim 41, wherein the mechanical wave comprises a plurality of waves having different frequencies and/or amplitudes.

49. The system of claim 41, wherein the mechanical wave generator comprises a frequency modulator for temporally modulating the frequency of the mechanical wave throughout the measuring of the electrical potential over time.

50. The system of claim 41, wherein the mechanical wave generator comprises an amplitude modulator for temporally modulating the amplitude of the mechanical wave throughout the measuring of the electrical potential over time.

51. The system of claim 41, wherein the working electrode is positioned in the liquid solution at a distance of about 1-200 micron from the solid surface.

52. The system of claim 41, further comprising a reference electrode.

53. The system of claim 52, wherein the reference electrode is positioned in the liquid solution further away from the solid surface than the working electrode, and at least about 200 micron away from the solid surface.

54. A method for identifying one or more ions in a liquid solution, the method comprising:
a) contacting the solution with a solid surface;
b) generating, by a mechanical wave generator configured to be in contact with the solid surface and/or with the liquid solution, a mechanical wave including at least one frequency and at least one amplitude, which propagates and excites ions in an electrical double layer (EDL) formed at an interface between the surface and the solution, thereby resulting in the EDL emanating an electrical field away therefrom;
c) measuring, by a working microelectrode configured to be positioned in the liquid solution in close proximity to the solid surface without contacting it, for each frequency and/or amplitude in the mechanical wave, a voltage of the electrical field emanated from the EDL over time; and
d) analyzing, by a computation module in a processor, a spectrum of the voltage measured for frequencies and/or amplitudes included in the mechanical wave to identify the one or more ions and concentrations thereof in the solution.

55. The method of claim 54, wherein generating a mechanical wave in step (b) of claim 54 comprises temporally sweeping through a range or a plurality of frequencies, and for each frequency measuring in step (c) of claim 54 the voltage leaked from the EDL.

56. The method of claim 54, wherein analyzing the voltage spectrum in step (d) of claim 54 comprises identifying at least one peak intensity of the voltage spectrum that is induced by a certain wave frequency, and identifying the one or more ions based on the ion having a relaxation frequency, half a relaxation frequency, or a relaxation frequency multiplied by an integer, that is close to the certain frequency.

57. The method of claim 56, wherein analyzing the voltage spectrum in step (d) of claim 54 further comprises identifying a series of peak amplitudes or intensities of the voltage spectrum, including a main peak at a wave frequency corresponding to a leading ion resonance harmonic and at least one smaller peak corresponding to secondary harmonics.

58. The method of claim 54, further comprising adding at least one known ion at a known concentration to the liquid solution prior to generating the mechanical wave in step (b) of claim 54.

59. The method of claim 54, further comprising performing steps (a)-(c) of claim 54 on at least one control solution including at least one known ion at a known concentration, and wherein the analyzing in step (d) of claim 54 comprises adjusting the voltage spectrum obtained for the liquid solution based on the voltage spectrum obtained for the control solution.

60. The method of claim 54, wherein the one or more ions comprise at least one ion present at a concentration of at least about 1 ppm.
